(19) United States
(12) Patent Application Publication
(10) Pub. No.: US 2015/0367116 A1
(43) Pub. Date: Dec. 24, 2015

(54) Applicator Tool

(71) Applicant: Kristina T. Cash, Humble, TX (US)

(72) Inventor: Kristina T. Cash, Humble, TX (US)

(21) Appl. No.: 14/719,545

(22) Filed: May 22, 2015

Related U.S. Application Data
(60) Provisional application No. 62/015,914, filed on Jun. 23, 2014.

(51) Int. Cl. A61M 35/00

(52) U.S. Cl. CPC A61M 35/003

(57) ABSTRACT

Provided herein are applicator tools and devices for conveniently and evenly applying cream or ointment to the skin in a shorter time and with little or no mess than using fingers alone. This applicator tool comprises a body portion substantially in a shape of teardrop, an applicator portion formed on a proximal end of the body portion and a base portion formed at the distal end of the body portion. Generally, the body portion may be a grip. The applicator portion may be a spatulate shaped sheet. The base portion comprises means for securing the applicator tool on a surface.

### CROSS-REFERENCE TO RELATED APPLICATION

This nonprovisional application claims benefit of priority under 35 U.S.C. §119(e) of provisional application U.S. Ser. No. 62/015,914, filed Jun. 23, 2014, the entirety of which is hereby incorporated by reference.

### BACKGROUND OF THE INVENTION

1. Field of the Invention

The present invention relates generally to the field of applicator tool products. More specifically, the present invention relates to applicator tools and methods for applying various creams and ointments.

2. Description of the Related Art

Creams or ointments are commonly used to protect skin from irritation caused by moisture and stool. An example of such a cream is a diaper cream typically applied to a baby.

The primary purpose of diaper cream is to prevent and/or treat skin irritation commonly referred to as diaper rash. Currently, the most common method for applying diaper cream is to use one's fingers to spread the cream onto the skin. As anyone who has ever applied diaper cream to a baby can attest, there are several drawbacks with this approach. Firstly, the person applying the ointment has diaper cream directly on their fingers. Therefore, most creams or ointments advise user to wash hands prior to application to prevent the transfer of germs. However, it is often difficult to conveniently find a place to wash hands at the moment of applying diaper creams. Thus, a germ-free applicator seems to be a solution for this problem. Also, applying diaper cream by fingers can create an unintended mess in the event that his/her hands are needed for other purposes such as such as holding the baby's legs up, or keeping a baby from rolling off from the changing station. Secondly, it is difficult to ensue that the diaper cream is spread evenly and effectively onto the skin. This is in part due to the fact that the average index finger for adults is less than 2 cm wide, resulting in the need for multiple applications to cover the entire surface area. Thirdly, cleansing the diaper cream from ones' fingers is often time consuming. Diaper cream is difficult to wash off given that its primary function is to repel moisture. Even once the cream has been removed from one's fingers, the smell of the cream often remains.

Other products have attempted to resolve some of these challenges but have resulted in new unaddressed challenges. As an example, one product consists of a container of diaper balm which can be rolled onto the skin like a very large tube of Chap Stick. The primary challenge associated with this product is that users must use the provided cream and do not have autonomy to use any cream of their choosing. A separate product patent exists on a brush head feature which attaches to diaper cream tube openings. In this case, users squeeze the tube of cream which pushes the product through the attached brush head. Challenges with this product include the ability to clean the product effectively after each use as well as the inability to use it with some cream containers.

Thus, there is a recognized need, for an applicator tool that is easy to clean, able to stand upright to avoid transferring ointment onto other surfaces, and configured to evenly apply creams or ointment on skin without occupy the user's both hands. The prior art is deficient in this respect. The present invention fulfills this long standing need and desire in the art.

### SUMMARY OF THE INVENTION

The present invention is directed to an applicator tool. The applicator tool comprises a body portion, an applicator portion formed at a proximal end of the body portion and a base portion formed at a distal end of the body portion.

The present invention also is directed to a device for applying a cream or ointment to a body. The device comprises a grip substantially in a shape of teardrop, an applicator affixed to a proximal end of the grip and a base affixed to a proximal end of the grip.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 is a front view of the applicator tool of the present invention.

FIG. 2 is a side view of the applicator tool of the present invention.

FIG. 3 is a perspective view of the applicator of the present invention, depicting the suction cup base of the applicator tool.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “proximal end” refers to the end toward the applicator portion of the tool. The term “distal end” refers to the end away from the applicator portion of the tool.

In one embodiment of the present invention, there is provided an applicator tool. The applicator tool comprises a body portion; an applicator portion formed at a proximal end of the body portion; and a base portion formed at a distal end of the body portion. In this embodiment, the body portion may be substantially teardrop shaped. The body portion may comprise silicone, plastic, nylon, resin, wood, metal or a combination thereof. The body portion may have a length of about 80 mm to about 200 mm. In a preferred embodiment, the body portion of the tool is a grip.

Also in this embodiment, the applicator portion may comprise a smooth spatulate sheet. The spatulate-shaped applicator portion may have tapered sides. The applicator portion may have a length of about 36 mm and a width of about 30.5 mm wide. Representative materials from which applicator portion can be made, may be, but are not limited to, silicone, silicone, plastic, nylon, resin, wood, metal, or a combination thereof. Further in this embodiment, the base portion of the applicator tool may comprise a suction cup disposed for attachment of the applicator tool to a flat surface.

In another embodiment of the present invention, there is provided a device for applying a cream or ointment to a body comprising a grip substantially in a teardrop shape; an applicator affixed to a proximal end of the grip; and a base affixed to a distal end of the grip. In a preferred embodiment, the grip has a length of about 80 mm to about 200 mm.

Also in this embodiment, the body portion or grip is made of a material such as, include but are not limited to, silicone, plastic, nylon, resin, wood, metal or a combination thereof. The applicator is spatulate shaped. The spatulate-shaped applicator has tapered sides. Applicator of the device may be made of materials such as, including but not limited to, silicone, plastic, nylon, resin, wood, metal or a combination thereof. Further in this embodiment, the applicator is about 26 mm long and about 30.5 mm wide. Further still in this embodiment, the base of the device comprises means for securing the device to a surface. The securing means comprise a suction cup, a clip, a clamp or a hook. In a preferred embodiment, the grip, applicator and securing means are formed as a unitary object.

Provided herein are applicator tools and devices for the even application of a cream or lotion, such as, but not limited to, a diaper cream, to a baby's skin without occupying both hands of the user. The applicator tool or device comprises a soft flexible applicator or applicator portion, a grip or body portion, and a base or base portion, such as a suction cup. The exterior of the applicator tool comprises a soft material such as silicone for comfortable gripping and gentle touching on the baby's skin.

The applicator or applicator portion is about 26 mm tall and about 30.5 mm wide. The applicator picks up the cream and transfers the cream from the tub or jar onto the skin. Users may place the desired amount of cream onto the applicator by dipping the applicator portion into a jar of cream or by squeezing cream from a tube onto the applicator. The body portion or grip is about 94 mm long, which allows the user to hold the applicator tool and to keep the fingers from coming into contact with the cream to stay clean. Once the cream is transferred to the applicator, the user holds the body portion or grip and applies the cream to the skin using brushing or sweeping motions. When the user finishes applying the cream, the applicator tool can be securely attach to most surfaces, such as a table, a wall etc., using the suction cup on the base of the applicator tool.

The diameter of the body portion or grip increases towards the base of the tool and decreases just under the applicator portion, i.e., has a substantially teardrop shape. The center of gravity of the applicator tool is located near the bottom of the body portion. The body portion or grip may comprise various materials such as plastic, silicone, wood, or metal.

As described below, the invention provides a number of advantages and uses, however such advantages and uses are not limited by such description. Embodiments of the present invention are better illustrated with reference to the Figure(s), however, such reference is not meant to limit the present invention in any fashion. The embodiments and variations described in detail herein are to be interpreted by the appended claims and equivalents thereof.

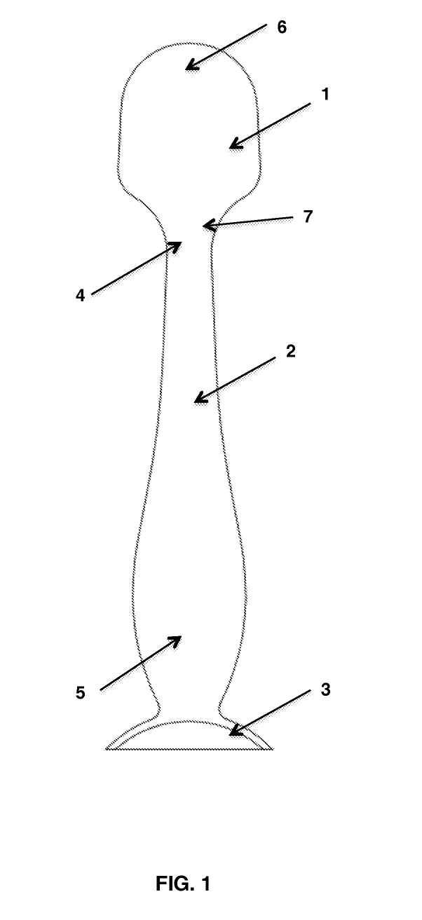

FIG. 1 is a line drawing for the front view of the applicator tool or device comprising an applicator portion 1, a body portion or grip 2 and a base portion or base 3. The applicator portion 1 comprises a smooth spatulate sheet about 26 mm long and about 30.5 mm wide with a curved top side. The body portion 2 has a substantially teardrop shape with a narrower proximal end 4 and a wider distal bottom 5. The applicator tool or device is a unitary object where the applicator portion 1 is formed from the proximal end 4 of the body portion and the base portion 3 is formed from the distal bottom 5 of the body portion. The base portion 3 may be a suction cup configured to secure the applicator tool on most types of surfaces. Alternatively, the base portion 3 may be other securing means the applicator tool, such as a clip, a hook, a tie or a clamp.

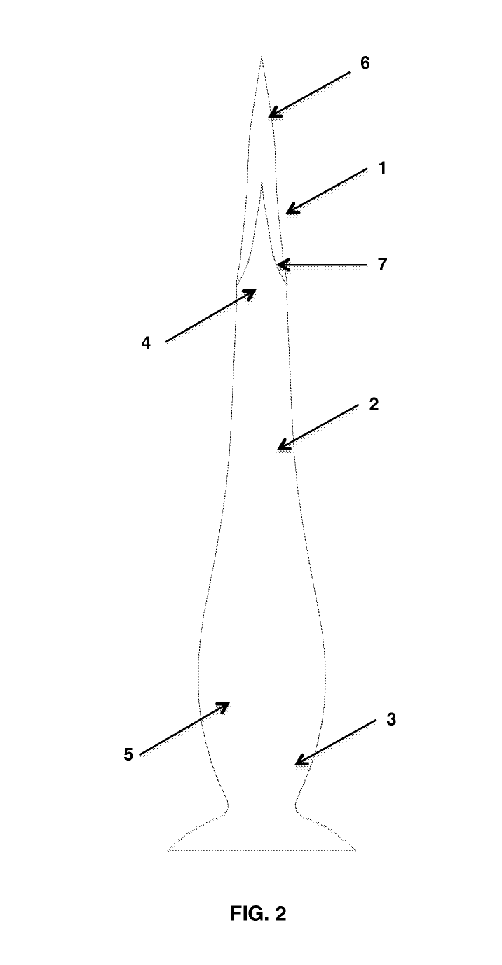

FIG. 2 is a side view perspective of the applicator tool, depicting the applicator portion 1 as a thin spatulate shaped sheet. The proximal end of the sheet 6 is tapered thinner than the distal end 7 thereof. Since the body portion is substantially teardrop shaped, the right and left side views and the front and back views of the body portion 1 and the front and back views of the body are identical. Moreover, the right and left sides of the applicator tool are mirror images as are the front and back of the applicator tool. The base portion 3, such as a suction cup may be substantially circular.

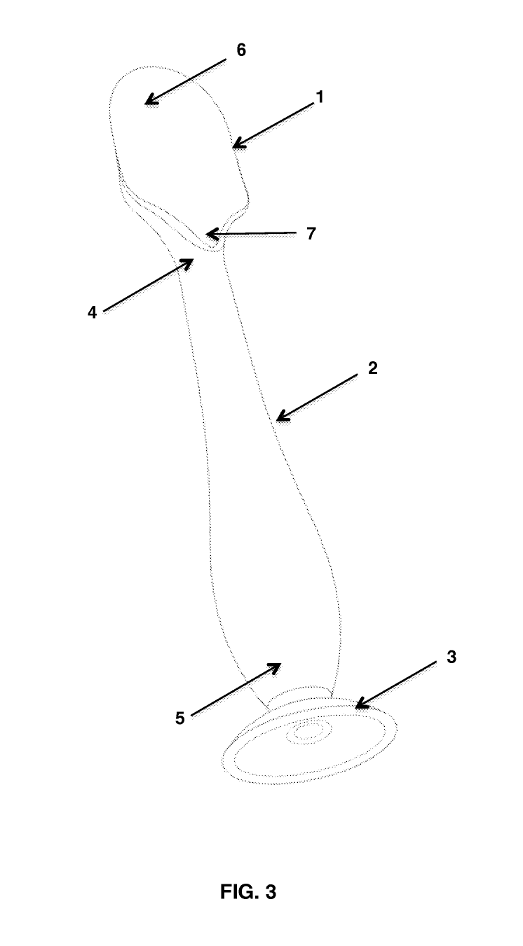

FIG. 3 is a line drawing of the perspective view of the applicator tool, showing the teardrop shaped body portion or grip 2, the applicator 1 and a circular suction cup as the base portion 3. The spatulate shaped applicator 1 has a thicker distal end 7 tapering toward the thinner proximal end 6.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention.

What is claimed is:

1. An applicator tool, comprising:
a body portion;
an applicator portion formed at a proximal end of said body portion; and
a base portion formed at a distal end of the body portion.

2. The applicator tool of claim 1, wherein said body portion is substantially teardrop shaped.

3. The applicator tool of claim 1, wherein said body portion comprises silicone, plastic, nylon, resin, wood, metal or a combination thereof.

4. The applicator tool of claim 1, wherein said body portion has a length of about 80 mm to about 200 mm.

5. The applicator tool of claim 1, wherein said body portion is a grip.

6. The applicator tool of claim 1, wherein said applicator portion comprises a smooth spatulate sheet.

7. The applicator tool of claim 6, wherein said spatulate-shaped applicator portion has tapered sides.

8. The applicator tool of claim 1, wherein said applicator portion has a length of about 36 mm and a width of about 30.5 mm.

9. The applicator tool of claim 1, wherein said applicator portion comprises silicone, plastic, resin, nylon, wood, metal, or a combination thereof.

10. The applicator tool of claim 1, wherein said base portion comprises a suction cup disposed for attachment of said applicator tool to a flat surface.

11. A device for applying a cream or ointment to a body, comprising:
a grip substantially in a teardrop shape;
an applicator affixed to a proximal end of the grip; and
a base affixed to a distal end of the grip.

12. The device of claim 11, wherein said grip has a length of about 80 mm to about 200 mm.

13. The device of claim 11, wherein said grip comprises silicone, plastic, nylon, resin, wood, metal or a combination thereof.

14. The device of claim 11, wherein said applicator is spatulate shaped.

15. The device of claim 14, wherein said spatulate-shaped applicator has tapered sides.

16. The device of claim 11, wherein said applicator comprises silicone, plastic, nylon, resin, wood, metal or a combination thereof.

17. The device of claim 11, wherein said applicator is about 26 mm long and about 30.5 mm wide.

18. The device of claim 11, where said base comprises means for securing said device to a surface.

19. The device of claim 18, wherein said securing means comprises a suction cup, a clip, a clamp or a hook.

20. The device of claim 11, wherein said grip, applicator and securing means are formed as a unitary object.
